(19) United States
(12) Patent Application Publication
(10) Pub. No.: US 2024/0342062 A1
(43) Pub. Date: Oct. 17, 2024

(54) IMPROVEMENTS IN OR RELATING TO ORGANIC COMPOUNDS

(71) Applicant: Givaudan SA, VERNIER (CH)

(72) Inventors: Justin WHITFIELD, Billerica, ME (US); John C WARNER, Wilmington, MA (US); Frederick Richard KEARNEY, Walpole, MA (US)

(21) Appl. No.: 18/034,617

(22) PCT Filed: Nov. 23, 2021
(86) PCT No.: PCT/EP2021/082589

§ 371 Date: Apr. 28, 2023

Related U.S. Application Data
(60) Provisional application No. 63/118,124, filed on Nov. 25, 2020.

(51) Int. Cl. A61K 8/11; A61K 8/33; A61K 8/64; A61K 8/73

(52) U.S. Cl. CPC A61K 8/11; A61K 8/33; A61K 8/645; A61K 8/731

(57) ABSTRACT

Disclosed is an encapsulated composition comprising at least one core-shell microcapsule. The at least one core-shell microcapsule comprises a core comprising at least one benefit agent and a shell surrounding the core. The shell comprises a first and a second polyelectrolyte which form a complex coacervate. The microcapsule comprises at least one interfacial enabler.

The present invention relates to an encapsulated composition, to a process for preparing an encapsulated composition, to an encapsulated composition obtainable by such a process, to a use of an interfacial enabler for obtaining an encapsulated composition and to a use of an encapsulated composition for obtaining a consumer product.

It is known to incorporate encapsulated benefit agents in consumer products, such as household care, personal care and fabric care products. Functional materials include for example fragrances, cosmetic actives, and biologically active ingredients, such as biocides and drugs. For avoidance of any doubt, the expressions “benefit agent” and “functional material” are herein used as synonyms.

Microcapsules that are particularly suitable for delivery of such benefit agents are core-shell microcapsules, wherein the core comprises the functional material and the shell is impervious or partially impervious to the core. Usually, these microcapsules are used in aqueous media and the encapsulated benefit agents are hydrophobic. A broad selection of shell materials can be used, provided this shell material is impervious or partially impervious to the encapsulated functional material.

Among the benefit agents, fragrances are encapsulated for a variety of reasons. Microcapsules can isolate and protect the fragrances from external suspending media, such as consumer product bases, with which they may be incompatible or unstable in. They are also used to assist in the deposition of fragrance ingredients onto substrates, such as skin, hair, fabrics or hard household surfaces. They can also act as a means for controlling the spatio-temporal release of the fragrance.

Microcapsules containing a hydrophobic liquid core surrounded by a layer of cross-linked gelatin-based simple or complex coacervate are well known in the art. For example U.S. Pat. No. 6,045,835 discloses a method for encapsulating flavors or fragrances into microcapsules having a hydrogel shell comprising gelatin and an oil core. In U.S. Pat. No. 8,088,403 B2 gelatin is preferred over other proteins because of its good physical-chemical properties.

However, gelatin is a protein of animal origin, which is typically obtained from fish, pork, beef and/or poultry. Nowadays consumers are increasingly concerned about using materials obtained from animal origin, especially in cases the materials are used in body care applications.

It is therefore a problem underlying the present invention to overcome the above-mentioned shortcomings in the prior art. In particular, it is a problem underlying the present invention to provide protein-based encapsulated compositions of the above-mentioned kind, being made of materials from non-animal source. The capsules should show good stability during manufacture and storage as well as beneficial release properties in application. Furthermore, the compositions should be producible in an operationally safe, robust and cost-efficient process.

These problems are solved by the subject-matter of the independent claims.

In a first aspect, the present invention relates to an encapsulated composition comprising at least one core-shell microcapsule. The at least one core-shell microcapsule comprises a core comprising at least one benefit agent and a shell surrounding the core. The shell comprises a first and a second polyelectrolyte which form a complex coacervate. The microcapsule comprises at least one interfacial enabler.

In the present context, by “interfacial enabler” is meant a species which has the ability to interact with at least one of the polyelectrolytes at the phase interface of the core composition, in order to facilitate shell formation. Without being bound to any theory, this interaction can be through dispersion forces, electrostatic forces or hydrogen bonds. But also covalent bonds are encompassed by this term.

The core of the core-shell microcapsules is made of a core composition. The core composition is in particular essentially water-immiscible.

By “essentially water-immiscible” it is meant that, when the core composition admixed with water, even under intensive stirring, at least 95 wt.-%, preferably at least 99 wt.-%, of the core composition phase separates from the water phase, either immediately or progressively after the cessation of the stirring.

Preferably, the amount of core composition is lower than the amount of aqueous phase, so that, when emulsified with the aqueous phase, the core composition forms a dispersed phase in the aqueous phase, generally in the form of core composition droplets.

By “coacervate” it is meant polyelectrolyte-rich droplets coexisting with an aqueous, polyelectrolyte-poor continuous phase. The droplet agglomerate at interfaces to form an interfacial layer.

In the context of the present invention, the coacervate droplets agglomerate at the interface between the core composition and the aqueous phase. As a result, a stable core-shell capsule is formed, comprising a core composition droplet being surrounded by coacervate droplets. These stabilize the composition droplet and form a capsule wall.

By “complex coacervation” or “complex coacervate” is meant the formation of an interfacial layer comprising a mixture of polyelectrolytes.

The phenomenon of complex coacervation may be observed under a light microscope, wherein it is marked by the appearance of a ring around the core composition droplet. This ring consists of the aforementioned polyelectrolyte-rich phase that has a different refractive index than the surrounding aqueous phase.

The complex coacervation of two polyelectrolytes is generally induced by bringing the two polyelectrolytes to their isoelectric point, meaning the point where the net charge of the ensemble of both polyelectrolytes is zero or close to zero, inducing thereby complexation of the two polyelectrolytes. This may be achieved by changing the salt concentration or, in the case of a polyampholyte, such as proteins, by changing the pH of the medium.

It has been found that the efficiency of complex coacervation between two polyelectrolytes may be significantly improved by employing an interfacial enabler. In particular, this significant improvement makes it possible to obtain vegan protein-based microcapsules that are as performing as gelatin-based microcapsules. Furthermore, by using such an interfacial enabler, it is possible to obtain microcapsules having a volume mean diameter below 25 μm and even below 10 μm, which is otherwise difficult to realize.

In particular embodiments of the present invention, the core consists of a liquid core composition. The interfacial enabler is then soluble in the core composition or is derived from a material that is soluble in the core composition.

Preferably, the interfacial enabler is or is derived from a polyfunctional molecule, preferably a bifunctional molecule. Polyfunctional molecules have been found to be particularly suitable for facilitating shell formation.

Without being bound to any theory, if the interfacial enabler is or is derived from a polyfunctional molecule, it can be a cross-linker, cross-linking the first and/or second polyelectrolyte.

In order to undergo complex coacervation, it is preferable that the first and second polyelectrolytes have opposite electrical charges at a given pH. As mentioned hereinabove, complex coacervation occurs when the neat electrical charge of the ensemble of both polyelectrolytes is zero or close to zero.

In the following, the first and second polyelectrolytes are interchangeable, meaning there is no preeminence of the “first” wording over the “second” wording.

The first polyelectrolyte is preferably a polyampholyte. Polyampholytes have the advantage of having a variable electrical charge, depending on the pH. Varying the pH allows therefore controlling the state of the polymer in water, referred to herein after as “the aqueous phase”. Thus, depending on the pH the polymer may be more or less cationic, more or less anionic or even change from a cationic polyelectrolyte to an anionic polyelectrolyte and vice versa. The pH at which the polymer has an electrical charge of zero is referred to as isoelectric point.

In preferred embodiments, the isoelectric point of the polyampholyte is below pH 7, preferably below pH 6, more preferably below pH 5.5, even more preferably below pH 5.0. Under these conditions the polyampholyte undergoes an anionic to cationic transition, which may easily be triggered by using a conventional Brønstedt acid. Concomitantly, as mentioned hereinafter, the charge of the second polyelectrolyte is also controlled by the pH in this region. This situation is particularly favorable for complex coacervation.

In order to obtain slurries having practically acceptable amounts of microcapsules, it is preferable that the solubility of the first and second polyelectrolytes is higher than 5 wt.-%, preferably higher than 10 wt.-%, in water at pH 7±0.5 and at room temperature. In the context of the present invention the term “water” includes deionized and tap water.

In preferred embodiments of the present invention, the polyampholyte is a protein, in particular a protein originating from a vegan source. Proteins are very versatile polyampholites, owing to their chemical and conformational diversity, hydrophilic to hydrophilic balance and electrostatic properties.

Such attributes are especially favorable for complex coacervation. The fact that vegan proteins do not originate from animals is an advantage in the today societal and ethical context.

In particularly preferred embodiments, the protein originating from a vegan source is selected from soy proteins, pea proteins, rice proteins and hemp proteins, preferably soy proteins. These proteins may be used as such or in denaturated form. Protein denaturation usually involves changes in the secondary, tertiary and quaternary structure of the protein, transforming a highly functional and specialized macromolecule into a material that can be employed in a broad range of applications. It is well known that denaturation may be induced by the action of, for example, temperature, pH, ionizing radiation, shear stresses, water structure destroying agents and detergents. The proteins may be isolated from the vegan source by known processes, such as extraction and centrifugation.

Protein isolates may contain materials that are not soluble in water and that may precipitate in or confer a turbid aspect to aqueous solutions comprising the protein. In particular embodiments of the present invention, aqueous solutions comprising a nominal percentage of the protein, in particular the protein originating from a vegan source, of 5 wt.-% comprise less than 0.1 wt.-%, preferably less than 0.05 wt.-%, of insoluble material, based on the total weight of the solution. Under “nominal percentage” is meant the weighed-in amount of protein added to the aqueous phase at the beginning of the dissolution process.

As an alternative to protein isolates, which generally have a protein content of 80-95 wt.-%, also protein concentrates with a lower protein content (e.g. about 70 wt.-%) may be used. However, it is preferable to use protein isolates in order to minimize the potential for interference from impurities in the encapsulation process. If protein concentrates are used, aqueous solutions comprising a nominal percentage of the protein, in particular the protein originating from a vegan source, of 5 wt.-% comprise less than 0.2 wt.-%, preferably less than 0.1 wt.-%, of insoluble material, based on the total weight of the solution.

In preferred embodiments of the present invention, the second polyelectrolyte is a polysaccharide, preferably a polysaccharide comprising carboxylic acid groups. Beyond the fact that polysaccharides are also bio-sourced and vegan, which is a considerable advantage with respect to the present invention, polysaccharides comprising carboxylic groups are particularly suitable to complex coacervation-mediated microencapsulation. On one hand, these polymers may have the ability to accumulate to interfaces and act as emulsifiers or co-emulsifiers. On the other hand, their carboxylic groups may be more or less deprotonated, depending on the pH, allowing the modulation of their anionic character and of their ability to establish electrostatic interactions with the protein.

The polysaccharide comprising carboxylic acid groups may comprise uronic acid units, in particular hexuronic acid units. Polysaccharides having uronic acid units, in particular hexuronic acid units, are broadly available in nature. The hexuronic acid units can be selected from the group consisting of galacturonic acid units, glucuronic acid units, in particular 4-O-methyl-glucuronic acid units, guluronic acid units and mannuronic acid units.

The polysaccharide comprising carboxylic acid groups may be branched. Branched polysaccharides comprising carboxylic acid groups have the advantage of forming more compact networks than linear polysaccharides and therefore may favor the imperviousness of the encapsulating shell, resulting in reduced leakage and greater encapsulation efficiency.

The carboxylic groups may also be grafted onto the polysaccharide by chemical means. For example carboxymethylcellulose may be obtained by reacting cellulose with chloroacetic acid or its sodium salt under alkaline conditions. In this reaction, the C2, C3 and C6 hydroxyl groups may be substituted with carboxyl function.

In preferred embodiments of the present invention, the polysaccharide comprising carboxylic acid groups is selected from the group consisting of carboxymethylcellulose, gum acacia, alginate, pectin, hyaluronic acid, xanthan gum, gellan gum, and their salts with monovalent alkaline metals.

Carboxymethylcellulose (CMC) and sodium carboxymethylcellulose are particularly preferred in the context of the present invention, because these modified celluloses are less prone to quality fluctuations, compared to unmodified polysaccharides. Moreover, the degree of substitution (DS) of CMC, meaning the average number of carboxylic acid or carboxylate groups divided by the number of glucopyranose units in the CMC macromolecule may be higher than the degree of substitution of other polysaccharide comprising carboxylic acid groups. For example, gum acacia consists of only 15 to 16% (DS=0.15-0.16) glucuronic acid, whereas in CMC one glucopyranose unit may contain up to 3 carboxylic groups, typically between 0.4 and 1.5 (DS=0.4-1.5). CMC having DS lower than 0.4 is sparingly solid, while higher grades are difficult to manufacture industrially.

Preferably, the molecular weight of the polysaccharide is selected in such a way that the polysaccharide macromolecules are large enough to form viscous coacervates and low enough to prevent the slurry of being too viscous.

In particularly preferred embodiments of the present invention, the carboxymethylcellulose and/or the sodium carboxymethylcellulose have a molecular weight of from 50′000 to 250′000 g/mol, preferably from 75′000 to 125′000 g/mol, and a degree of substitution of from 0.5 to 1.5, preferably from 0.6 to 1.1.

The ratio of the second polyelectrolyte to the first polyelectrolyte is determined by their respective electrostatic charge and the necessity to form a electrostatically neutral polymer-polymer complex within the complex coacervation pH range. This ratio is typically determined experimentally.

In case the first polyelectrolyte is a protein and the second polyelectrolyte is carboxymethylcellulose and/or sodium carboxymethylcellulose, then the weight ratio of the carboxymethylcellulose and/or the sodium carboxymethylcellulose to the protein, preferably the protein originating form a vegan source, more preferably the soy protein, is from 0.05 to 0.2, preferably from 0.08 to 0.12.

Preferably, the interfacial enabler is or is derived from a diacid or a dialdehyde, even more preferably from an aromatic dialdehyde. Whereas, diacids effectively improve encapsulation, the resulting microcapsules are less thermally stable than microcapsules obtained by using dialdehydes as interfacial enabler.

The dicarboxylic acid may be selected from 1,3-propanedicarboxylic acid (glutaric acid), 1,4-butanedicarboxylic acid (adipic acid), 1,5-pentanedicarboxylic acid (pimelic acid) and 1,4-cyclohexanedicarboxylic acid.

The cyclic dialdehydes may be selected from ortho-phtalaldehyde (1,2-benzenedicarbaldehyde), isophtalaldehyde (1,3-benzenedicarbaldehyde), terephtalaldehyde (1,4-benzenedicarbaldehyde), 1,2-cycloxanedicarbaldehyde, 1,3-cycloxanedicarbaldehyde, 1,4-cycloxanedicarbaldehyde, cyclopentane-1,3-dicarbaldehyde, [1,1′-biphenyl]-3,4′-dicarbaldehyde, [1,1′-biphenyl]-3,3′-dicarbaldehyde, [1,1′-biphenyl]-3,2′-dicarbaldehyde, 9,10-anthracenedicarbaldehyde, 4,4′-biphenyldicarbaldehyde, furan-2,5-dicarbaldehyde, and 1,4-bis(X-formyl-phenoxy) alkane, wherein X=2, 3 or 4 and the alkane residue includes 2 to 6 carbon atoms, more particularly 4 carbon atoms, 1,4-diformylpyperazin, and 2,2′-bipyridine-4,4′-dicarbaldehyde. More preferably, the cyclic dialdehyde is an aromatic dialdehyde selected from ortho-phtalaldehyde (1,2-benzenedicarbaldehyde), isophtalaldehyde (1,3-benzenedicarbaldehyde), terephtalaldehyde (1,4-benzenedicarbaldehyde), [1,1′-biphenyl]-3,4′-dicarbaldehyde, [1,1′-biphenyl]-3,3′-dicarbaldehyde, [1,1′-biphenyl]-3,2′-dicarbaldehyde, 9,10-anthracenedicarbaldehyde and 4,4′-biphenyldicarbaldehyde.

In preferred embodiments, interfacial enabler is or is derived from a dialdehyde selected from the group consisting of 1,3-benzenedicarbaldehyde, 1,3-cycloxanedicarbaldehyde, cyclopentane-1,3-dicarbaldehyde, furan-2,5-dicarbaldehyde and 1,4-diformylpyperazin, even more preferably from 1,3-benzenedicarbaldehyde and 1,4-diformylpyperazin, and still more preferably from 1,3-benzenedicarbaldehyde. These particular interfacial enablers are well soluble in the core composition and therefore particularly advantageous for providing suitable microcapsules according to the present invention.

In preferred embodiments of the present invention, the interfacial enabler is present at a level of from 0.1 to 5 wt.-%, preferably from 0.5 to 2.0 wt.-%, still more preferably from 0.75 to 1.5 wt.-%, based on the weight of the core composition. If the level of interfacial enabler is too low it becomes ineffective in forming microcapsules having the desired properties: The microcapsules obtained are too large and thermally unstable. On the other hand, increasing the level of the interfacial enabler beyond a certain level, for instance 2 wt.-%, does not improve the properties of the microcapsules and may be deleterious to the quality of the core composition. For example, if the core composition is a fragrance, then an excess of interfacial enabler may affect the olfactive profile of this fragrance. Furthermore, at higher levels, for instance more than 5%, solubility issues may be encountered.

Optimal performances, in terms of microcapsule diameter, encapsulation efficiency and microcapsule stability can be obtained when the weight ratio of the interfacial enabler to the protein, preferably a protein originating form a vegan source, even more preferably the soy protein, is from 0.01 to 0.1, preferably from 0.02 to 0.08, even more preferably from 0.03 to 0.07.

The benefit agent comprised in the core or the core composition can be selected from the group consisting of fragrance ingredients, cosmetic ingredients and biologically active ingredients.

In particular embodiments of the present invention, the core composition comprises at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated perfumes according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy) butan-2-olAMBERKETAL (3,8,8,11a-tetramethyldodecahydro-1H-3,5a-epoxynaphtho[2,1-c]oxepine); AMBERMAX (1,3,4,5,6,7-hexahydro-.beta.,1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino) benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl) propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl) quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4 (5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl) hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl)acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl) but-2-en-1-one); DAMASCONE ALPHA ((E)-1-(2,6,6-trimethylcyclohex-2-en-1-yl) but-2-en-1-one); DAMASCONE DELTA (1-(2,6,6-trimethyl-1-cyclohex-3-enyl) but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL (2-methyl-1-phenylpropan-2-yl acetate); DIMETHYL BENZYL ACETATE CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5 (6H)-ylidene) butanal); EBANOL ((E)-3-methyl-5-(2,2,3-trimethylcyclopent-3-en-1-yl) pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl) ethanone); FLORALOZONE (3-(4-ethylphenyl)-2,2-dimethylpropanal); FLORHYDRAL (3-(3-isopropylphenyl) butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl) pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS 3-(benzo[d][1,3]dioxole-5-carbaldehyde); HERBANATE ((2S)-ethyl isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); IRONE ALPHA ((E)-4-(2,5,6,6-tetramethylcyclohex-2-en-1-yl) but-3-en-2-one); ISO E SUPER (1-(2,3,8,8-tetramethyl-1,2,3,4,5,6,7,8-octahydronaphthalen-2-yl) ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl) phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl) but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl) methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy) hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl) ethanone); METHYL CINNAMATE (methyl 3-phenylprop-2-enoate); METHYL DIANTILIS (2-ethoxy-4-(methoxymethyl) phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl) propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl) propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl) butanamide); PEACH PURE (5-heptyldihydrofuran-2 (3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl) acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-PINENE BETA (6,6-dimethyl-2-trimethylbicyclo[3.1.1]hept-2-ene); methylenebicyclo[3.1.1]heptane); PINOACETALDEHYDE (3-(6,6-dimethylbicyclo[3.1.1]hept-2-en-2-yl) propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2 (3H)-one); RADJANOL SUPER ((E)-2-ethyl-4-(2,2,3-trimethylcyclopent-3-en-1-yl) but-2-en-1-ol); RASPBERRY KETONE (4-(4-hydroxyphenyl) butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl) pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl) ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl) pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl) propan-2-ol); TERPINEOL ALPHA (2-(4-methyl-1-cyclohex-3-enyl) propan-2-ol); TERPINEOL PURE (2-(4-methylcyclohex-3-en-1-yl) propan-2-ol); TERPINOLENE (1-methyl-4-(propan-2-ylidene)cyclohex-1-ene); TERPINYL ACETATE (2-(4-methyl-1-cyclohex-3-enyl) propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In particularly preferred embodiments of the present invention, more than 75 wt.-%, preferably more than 80 wt.-%, even more preferably more than 85 wt.-%, even still more preferably more than 90 wt.-%, even yet still more preferably more than 95 wt.-%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl) but-2-en-1-one); DAMASCONE ALPHA ((E)-1-(2,6,6-trimethylcyclohex-2-en-1-yl) but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl) pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl) butanal); FLORIDILE ((E)-undec-9-enenitrile); GALBANONE PURE (1-(5,5-dimethylcyclohex-1-en-1-yl) pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); ISOAMYL ACETATE (3-methylbutyl acetate); ISOAMYL BUTYRATE (3-methylbutyl ((E)-2-methoxy-4-(prop-1-en-1-yl) phenol); butanoate); ISOEUGENOL ISOJASMONE B 11 (2-hexylcyclopent-2-en-1-one); ISORALDEINE ((E)-3-methyl-4-(2,6,6-trimethylcyclohex-2-en-1-yl) but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MANZANATE 2(ethyl-2-methylpentanoate); MAYOL ((4-isopropylcyclohexyl) methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl) phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl) propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl) propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2 (3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2 (3H)-one); RASPBERRY KETONE (4-(4-hydroxyphenyl) butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl) propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These ingredients have the advantage of providing microcapsules from which not only the shell but also the core is biodegradable.

In context of the present invention, a “biodegradable ingredient” is an ingredient which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

For assessment of the pass criteria for “readily biodegradable”, the biodegradation study can be selected from the group consisting of OECD Method 301C, OECD Method 301D, OECD Method 301F and OECD Method 310. These methods are suitable for volatile materials.

OECD Method 301C, OECD Method 301D and OECD Method 301F are described in the OECD Guidelines for the Testing of Chemicals, Section 3, Test No. 301: Ready Biodegradability (Adopted: 17th July 1992; https://doi.org/10.1787/97892640703-(9-en).

OECD Method 310 is described in the OECD Guidelines for the Testing of Chemicals, Section 3, Test No. 310: Ready Biodegradability-CO2 in sealed vessels (Headspace Test) (Adopted: 23 Mar. 2006; Corrected: 26 Sep. 2014; https://doi.org/10.1787/978926-(016316-en).

In a particular aspect of the present invention, the pass criteria for “readily biodegradable” are assessed according to OECD Method 301F, which refers to manometric respirometry. In this method the pass level for “ready biodegradability” is to reach 60% of theoretical oxygen demand and/or chemical oxygen demand. This pass value has to be reached in a 10-day window within the 28-day period of the test. The 10-day window begins when the degree of biodegradation has reached 10% of theoretical oxygen demand and/or chemical oxygen demand and must end before day 28 of the test.

Given a positive result in a test of ready biodegradability, it may be assumed that the chemical will undergo rapid and ultimate biodegradation in the environment (Introduction to the OECD Guidelines for the Testing of Chemicals, Section 3, Part 1: Principles and Strategies Related to the Testing of Degradation of Organic Chemicals; Adopted: July 2003).

For assessment of the pass criteria for “inherently biodegradable”, the biodegradation study can be OECD Method 302C, but also OECD Method 301F can be used, although with different pass criteria. Also these methods are suitable for volatile materials.

OECD Method 302C is described in the OECD Guidelines for the Testing of Chemicals, Section 3, Test No. 302C: Inherent Biodegradability: Modified MITI Test (II) (Adopted: 12 May 1981; Corrected 8 Sep. 2009; https://doi.org/10.1787/9789264070-(00-en).

In a particular aspect of the present invention, the pass criteria for “inherently biodegradable” are assessed by OECD Method 302C. In this method the pass level for “inherently biodegradability” is then to reach 70% of theoretical oxygen demand. There is no time limit to reach this level.

Biodegradation rates above 70% may be regarded as evidence of inherent, ultimate biodegradability (OECD Guidelines for the Testing of Chemicals, Section 3, Part 1: Principles and Strategies Related to the Testing of Degradation of Organic Chemicals; Adopted: July 2003).

If OECD Method 301F is used for assessment of the pass criteria for “inherently biodegradable”, the pass level is 60% of theoretical oxygen demand and/or chemical oxygen demand. This pass value can be reached after the 28-day period of the test, which is usually extended to 60 days. No 10-day window applies.

In the present context, if an ingredient is an essential oil, it is considered to be a “biodegradable ingredient” if all of its constituents present at a level ≥1 wt.-% fall under the definition of “inherently biodegradable” and/or “readily biodegradable” as defined herein above. However, the essential oil can also be subjected to the above-mentioned biodegradation tests.

The core composition may also comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

The core composition may also comprise at least one functional cosmetic ingredient. The functional cosmetic ingredients for use in the encapsulated composition are preferably hydrophobic.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

In order to offer an optimal balance between stability, deposition on substrate and performance, the volume median diameter of the microcapsules (Dv (50)) can be from 1 to 150 μm, preferably from 5 to 100 μm, even more preferably from 8 to 25 μm. Microcapsules having diameters smaller than 5 μm show large surface to volume ratios and are therefore more prone to leaching, whereas, as the number of microcapsule decreases with increasing diameter, too large microcapsules may not be numerous enough to provide noticeable benefits. Furthermore, large microcapsules may be visible in the product or let visible stain on the substrate.

A particular aspect of the present invention relates to an encapsulated composition comprising at least one core-shell microcapsule. The at least one core-shell microcapsule comprises a core comprising at least one benefit agent and a shell surrounding the core. The shell comprises a first and a second polyelectrolyte which form a complex coacervate. The microcapsule comprises at least one cross-linker, cross-linking the first and/or second polyelectrolyte. The cross-linker is derived from a dialdehyde selected from the group consisting of 1,3-benzenedicarbaldehyde, 1,3-cycloxanedicarbaldehyde, cyclopentane-1,3-dicarbaldehyde, furan-2,5-dicarbaldehyde and 1,4-diformylpyperazin. Preferably, the cross-linker is derived from 1,3-benzenedicarbaldehyde. The first polyelectrolyte is a protein selected from the group consisting of soy proteins, pea proteins, rice proteins and hemp proteins. Preferably, the first polyelectrolyte is a soy protein.

In a second aspect, the present invention provides a process for preparing an encapsulated composition, in particular a composition as described herein above, the process comprising the steps of:
a) Providing a core composition comprising an interfacial enabler or a material from which an interfacial enabler may be derived;
b) Providing an aqueous phase comprising a first and a second polyelectrolyte;
c) Emulsifying the core composition provided in step a) into the aqueous phase provided in step b) in order to obtain core composition droplets having a volume median diameter of 1 to 100 μm, preferably 5 to 75 μm, more preferably 8 to 60 μm, even more preferably 7 to 20 μm, dispersed in the aqueous phase;
d) Decreasing the pH until the coacervation pH has been reached, forming thereby a slurry of core-shell microcapsules;
e) Optionally: Adding a cross-linking agent, a further cross-linking agent if applicable, and keeping the slurry under stirring, in order to obtain a slurry of cross-linked microcapsules.

In regard to step a), the mixing of the core ingredients may be performed at room temperature under stirring.

In regard to step b), the aqueous phase may be provided at a pH of from 5 to 13, preferably from 7 to 13, more preferably from 9 to 12.5, even more preferably from 11 to 12. The polyelectrolytes are better solubilized under such alkaline conditions. This step is optimally performed at 30° C. or more, but preferably less than 60° C., as proteins submitted to high temperature and high pH may be prone to hydrolysis. Optimally, the temperature of dissolution is 45±5° C.

Thus, in preferred embodiments of the present invention, the protein, preferably the protein originating form a vegan source, is dissolved in the aqueous phase at a temperature of 45±5° C., preferably at a pH of from 5 to 13, more preferably from 7 to 13, still more preferably from 9 to 12.5, even still more preferably from 11 to 12.

Furthermore, in preferred embodiments of the present invention, the polysaccharide is dissolved in the aqueous phase at a temperature of 45±5° C., preferably at a pH of from 5 to 13, more preferably from 7 to 13, still more preferably from 9 to 12.5, even still more preferably from 11 to 12.

In regard to step c), the emulsion may be obtained at a temperature of from 30 to 60° C., preferably from 30 to 40° C. In this temperature range, it is expected that the polymer-polymer complex is still sufficiently weak to allow easy disruption of the core/water interface and subsequent disruption of the core droplets.

Adding a water-soluble or water-dispersible cross-linking agent, a further cross-linking agent if applicable, at the end of the complex coacervation allows the formation of a covalently cross-linked network around the microcapsule core, forming thereby a cross-linked encapsulating shell. Cross-linking typically involves the free amine groups of the protein, which are the most reactive available groups in the complex coacervate. Suitable cross-linking agents include 1,3-propane dialdehyde, 1,4-butanedialdehyde, 1,5-pentanedialdehyde, or 1,6-hexanedialdehyde (glutaraldehyde), succinicdialdehyde, glyoxal, glyoxyl trimer, resorcinol, multi-functional N-hydroxysuccinimide, isocyanates, di-, tri- and polyanhydrides, diesterimides, carbodiimides and disuccinimidyl dicarboxylates.

Alternatively, protein-protein cross-linking may advantageously be promoted by adding transglutaminase to the slurry. Transglutaminase catalyzes the cross-linking between two protein (or peptide) segments by forming a covalent bond between the glutamine residue of one segment and the lysine residue of the second segment. If transglutaminase is employed for protein-protein cross-linking, it is preferred to use a protein with higher amounts of the amino acids glutamine and lysine. Such proteins are for instance nut and legume globular proteins, and in particular soy protein.

In preferred embodiments of the present invention, in step e), the cross-linking agent, the further cross-linking agent if applicable, is added at a temperature of from 10° C. to 50° C., more particularly from 15° C. to 40° C., and the reaction is conducted over 1 to 20 hours, preferably from 5 to 15 hours, for example overnight, before the slurry is let cool at room temperature.

In particular embodiments of the present invention, the cross-linking agent, the further cross-linking agent if applicable, is selected from the group consisting of glutaraldehyde and transglutaminase.

Furthermore, the slurry of microcapsules obtained in step e) may be dried by any means known to the art, such as oven drying, drum drying, lyophilisation, spray drying or fluid bed drying. The drying process may be preceded by the separation of the microcapsules from the slurry by flotation.

Preferably, dehydration is performed by using spray drying or multistage spray drying. Typically, spray drying is performed by spraying the slurry obtained in step d) or e) in a hot chamber by using a nozzle, preferably a two-fluid nozzle, or a rotary atomizer or spinning disk. Typically, the slurry may be pumped and atomized through the nozzle or spinning disk with a co-current air flow in the spray drying apparatus for drying. A typical inlet temperature might be set at from 160 to 220° C., and an outlet temperature set at from 60 to 100° C. Dry particles comprising enrobed microcapsules and having particle sizes of about 10 to about 120 μm may then be collected at the dryer outlet. Such drying method is well known to the art. In a multiple stage dryer, the spray-dried particles are further agglomerated in order to obtain larger particles having particle sizes of from 100 to 250 μm.

In alternative embodiments of the present invention, the slurry obtained in step d) or e) may be sprayed on a fluidized carrier material having a pre-defined average particle size in a fluid bed dryer, such as a Wurster coating equipment in order to obtain carrier particles coated with a dry layer of microcapsules.

In further alternative embodiments of the present invention, the microcapsules comprised in the slurry obtained in step d) or e) may be granulated or agglomerated in a fluid bed dryer, in order to obtain particles having particle sizes of from 150 to 400 μm or more.

In other alternative embodiments of the present invention, the microcapsules may be separated from the slurry by flotation or filtration and dried in a fluid bed dryer at a temperature of from 60 to 150° C., more particularly from 80 to 120° C., in order to obtain particles having about the same size as the microcapsules comprised in the slurry obtained in step d) or e).

The method according to the present invention may also additionally comprise a dilution step, wherein the dried particles are admixed with at least one carrier material selected from the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides, polyethylene glycol, polyvinylpyrrolidone, citric acid or any water soluble solid acid, fatty alcohols, fatty acids.

Admixing the dried microcapsules with the at least one carrier material may be performed by any means known to the art, including convective blending, blade blending, fluidization blending, tumble blending and vortex blending. In a third aspect, the present invention provides an encapsulated composition obtainable by the process described herein above.

In a fourth aspect, the present invention provides a use of an interfacial enabler for obtaining an encapsulated composition, in particular an encapsulated composition as described herein above.

Furthermore, the present invention provides a consumer product comprising an encapsulated composition as described herein above, preferably a fabric care product, a home care product or a personal care product.

The encapsulated compositions of the present invention may be used to perfume all manners of consumer products, including laundry care detergents, laundry care conditioners, fabric refreshers, personal care cleansing compositions, such as shampoos, bath and shower gels, liquid soaps, soap bars, personal care conditioning compositions, such as hair care conditioners, bath and shower lotions, deodorant compositions, antiperspirant compositions, home care compositions, such as hard surface cleaners, and heavy duty detergents.

The consumer products according to the present invention may be used for treating substrates, such as fabrics, skin, hair, animate and inanimate surfaces, hard surfaces, wherein the action of treating a substrate includes washing, cleansing, softening, caring, finishing, scenting and/or deodorizing this substrate.

In one aspect of the present invention, a consumer product contains the compositions as described herein above, preferably at a level of 0.005 to 5 wt.-%, more preferably from 0.01 to 1 wt.-%, and still more preferably from 0.02 to 0.5 wt.-%, of the consumer product.

In many cases, the consumer products according to the present invention contain surfactants, such as anionic, cationic, amphoteric or non-ionic surfactants.

The consumer products according to the present invention may contain acids or bases, or substances providing acidity or alkalinity, also referred to as acidity sources or alkalinity sources.

The consumer products according to the present invention may contain builders for reducing water hardness, such as phosphates, polyphosphates, polycarboxylates, sodium citrate, sodium carbonate, sodium silicate, sodium aluminosilicate (zeolite).

In many cases, the consumer products according to the present invention are liquid and may contain further additives, such as solvents, fillers, texturing agents, such as thickener and rheological aids, distributing aids, anti-redeposition agents, preservative agents, deodorizing agents, cosmetic ingredients, and surface enhancing agents.

A consumer product containing microcapsules of the present invention may contain at least one solvent selected from water-soluble solvents, or water-insoluble, or partially water-soluble solvents.

A consumer product containing microcapsules of the present invention may contain at least one texturing agent and/or colloid stabilizer, selected from rheology modifiers, thickener, gel-forming agents, thixotropic agents, and dispersing agents.

A consumer product containing microcapsules of the present invention may contain at least one silicone, selected from, but not limited to dimethicone, poly(dimethylsiloxabedimethylsiloxane), amino-silicone, such as amodimethicone, trialkylammonium-silicone salts, ethoxylated silicones.

A consumer product containing microcapsules of the present invention may contain at least one cosmetic ingredient selected from, but not limited to emollients, moisturizing agents, anti-wrinkle agents, exfoliating agents, sunscreen agents, dyes, pigments, talcum, conditioning agents, hair styling agents, and antidandruff agents.

A consumer product containing microcapsules of the present invention may contain at least one fabric enhancing agent, selected from, but not limited to softening agents, optical brighteners and antistatic agents.

In particular embodiments of the present invention, the consumer product is a personal care deodorant or an antiperspirant composition that can be in the form of a sprayable composition or a composition that can be applied directly on skin by means of a roll-on device, a stick, a cream or a powder. The encapsulated composition according to the present invention may be incorporated in the deodorant or antiperspirant composition as a slurry of microcapsules, as obtained by the process described hereinabove, or in dry form.

Sprayable deodorant and antiperspirant compositions may comprise a condensed propellant gas, such as iso-butane; suspending agents, such as silica and hydrophobic bentonites; one or more solvents, such as water and ethanol; lipids, such as isopropyl myristate; surface active agents; polyols, such as glycerol; antiperspirant compounds, such as aluminium chlorohydrate; deodorizing compounds, such zinc carbonate, cyclodextrins, as well as calcium, aluminium, magnesium or zinc salts of unsaturated aliphatic hydroxycarboxylic acids, such as zinc ricinoleate; and perfume.

Deodorant and antiperspirant roll-on compositions may be in the form of gels or liquids comprising gelling agents, such as sodium salt of fatty acids, for example sodium stearate, polyamides, and silicone-based gelling agents; suspending agents, such as hydrophobic bentonite; emulsifiers, such as fatty acid polyethyleneglycol esters, for example Steareth-2, Steareth-4, Steareth-6, Steareth-7, Steareth-10, Steareth-11, Steareth-13, Steareth-15, Steareth-20, and glycerol esters and ethers; fatty alcohols, such as cetyl alcohol and stearyl alcohol; antiperspirant compounds, such as aluminium chlorohydrate, lipids, such as isopropyl myristate and vegetable oils; fatty acids, such as stearic acid; alcohols and polyols, such as ethanol and glycerol; and water.

Deodorant sticks and creams may comprise antiperspirant complexes, such as aluminum and aluminum zirconium polychlorohydrates waxy materials, such as stearyl alcohol, cetyl alcohol, admixed with hydrogenated oils, such as hydrogenated castor oil; emulsifiers, such as glyceryl stearate;

emollients, such as cyclodimeticone; and powder materials, such as talc and starch.

In a fourth aspect, the present invention relates to a use of an encapsulated composition as described herein above for obtaining a consumer product.

Further features and particular advantages of the present invention become apparent from the following examples.

### EXAMPLE 1: PREPARATION OF MICROCAPSULES

In Example 1.1, microcapsules according to the present invention were prepared by performing the steps of:
a) Providing a core composition by dissolving 0.25 g of isophthalaldehyde (1,3-benzenedicarbaldehyde) and 0.004 g of Solvent Yellow 98 fluorescent dye in 19.75 g of a fragrance composition;
b) Providing a first aqueous phase prepared by dissolving 4.55 g of solid soy protein isolate (Clarisoy 170, ex Arthur Daniel Midland Company) in 41.7 g of a 0.43 wt.-% sodium hydroxide solution in deionized water (pH of 11.5) at a temperature of 45° C. and letting the solution cool to room temperature;
c) Providing a second aqueous phase prepared by dissolving 0.45 g of carboxymethylcellulose in 19.3 g of a 0.6 wt.-% sodium hydroxide solution in deionized water (pH of 11.5) at a temperature of 45° C. and letting the solution cool to room temperature;
d) Providing a third aqueous phase consisting of a sodium hydroxide solution in deionized water at pH 11.5;
e) Heating up the first aqueous phase to 35° C. in a 400 ml reactor vessel under stirring by using a stir paddle;
f) Adding dropwise the second aqueous phase to the first aqueous phase under stirring, over a period of 15 minutes, through an addition funnel;
g) Rinsing the addition funnel with about 1 g of the third aqueous phase and collecting this rinse water in the reactor;
h) Increasing the stirring rate to 300 rpm and adding dropwise the core composition through an addition funnel over a period of 24 minutes, in order to obtain an emulsion;
i) Rinsing the addition funnel with about 1 g of the third aqueous phase and collecting this rinse water in the reactor;
j) Reducing the stirring speed to 180 rpm and adding the rest of the third aqueous phase over 13 minutes through the addition funnel, while keeping the emulsion obtained in step i) under stirring;
k) Adding a 22.5 wt.-% aqueous solution of citric acid continuously to the emulsion obtained in step j) at a rate of 1.3 ml/hour until the coacervation pH of 5.2±0.2 has been reached, forming thereby a slurry of core-shell microcapsules;
l) Slowly adding 1.3 ml of a 25 wt.-% glutaraldehyde solution in water;
m) Keeping the slurry formed in step l) under stirring for 60 minutes at 35° C. and then letting the slurry cool to room temperature overnight (about 15 hours), in order to obtain a slurry of cross-linked microcapsules.

The solid content, expressed as weight percentage of the initial slurry deposited on a thermo-balance operating at 120° C., was taken at the point where the drying-induced rate of weight change had dropped below 0.1 wt.-%/min. The ratio of the measured solid content to the theoretical solid content, calculated based on the weight of fragrance composition and encapsulating materials used, was taken as a measurement of encapsulation efficiency, expressed in wt.-%.

The microcapsule size distribution was measured using a Beckman-Coulter LS 13 320 laser particle size analyzer.

The slurry was free of agglomerates, the solid content of the slurry obtained was 20.2 wt.-%, the volume median size Dv (50) was 9 μm, the Dv (90) was 15 μm and the encapsulation efficiency was 95 wt.-%.

In Example 1.2, the microcapsules according to the present invention were produced by the same process as in Example 1.1, but glutaraldehyde was replaced in step l) by 0.4 g of transglutaminase (Activa TI, ex Ajinomoto), dissolved in 3.6 ml of deionized water. In this case, in step m), the temperature of the slurry was increased to 50° C. for 1 hour and then reduced to 35° C. and maintained at this latter temperature overnight (about 16 to 17 hours) before the slurry of cross-linked microcapsules was let cool to room temperature.

The slurry was free of agglomerates, the solid content of the slurry obtained was 20.6 wt.-%, the volume median size Dv (50) was 6 μm, the Dv (90) was 10 μm and the encapsulation efficiency was 96 wt.-%.

### EXAMPLE 2: VARIATION OF ENABLER

In Examples 2.1 to 2.16, microcapsules according to the present invention were prepared by performing the steps described in Example 1.1 or 1.2, but with various enablers and enabler concentrations. The details of these experiments are reported in Table 1 for samples of microcapsules cross-linked with glutaraldehyde and in Table 2 for samples of microcapsules cross-linked with transglutaminase, together with the microcapsule size, encapsulation efficiency and heptane extractable oil. The percentage of enabler refers to the total weight of the core composition.

The heptane extractable oil was determined as follows: 1.5 g of slurry was extracted with 5.5 ml of heptane for 2 hours under tumbling at 30-40 rpm. This mixture was then centrifuged at 3150 rpm for 5 minutes. The upper heptane phase was siphoned off and its VIS spectrum between 350 nm and 650 nm was recorded. The percentage of extracted oil was calculated from the absorbance at a wave length of 449 nm, based on a linear calibration curve.

TABLE 1
Samples cross-linked with glutaraldehyde

| | | Enabler | Dv(50) | Encapsulation | Extracted oil |
|---|---|---|---|---|---|
| | | concentration | Dv(90) | efficiency | with heptane |
| Example | Enabler | [wt.-%] | [μm] | [wt.-%] | [wt.-%] |
| 2.1 | — | 0 | 23 | 78 | n.d. |
| | | | 46 | | |
| 2.2 | isophtalaldehyde | 0.25 | 9 | 97 | 2.3 |
| | | | 16 | | |
| 2.3 | isophtalaldehyde | 0.50 | 9 | 95.8 | 2.7 |
| | | | 15 | | |
| 2.4 | isophtalaldehyde | 0.75 | 8 | 95.7 | 2.5 |
| | | | 13 | | |
| 2.5 | isophtalaldehyde | 1.0 | 9 | 94.6 | 2.2 |
| | | | 14 | | |
| 2.6 | isophtalaldehyde | 1.25 | 9 | 94.5 | 0.7 |
| | | | 15 | | |
| 2.7 | 1,4-diformyl- | 1.25 | 7 | 95.0 | n.d. |
| | piperazine | | 29¹ | | |
| 2.8 | pimelic acid | 1.5 | 24 | 57.0 | <3.0 |
| | | | 46 | | |

¹Larger capsules in the 50 to 100 μm were also present.

TABLE 2
Samples cross-linked with transglutaminase

| | | Enabler | Dv(50) | Encapsulation | Extracted oil |
|---|---|---|---|---|---|
| | | concentration | Dv(90) | efficiency | with heptane |
| Example | Enabler | [wt.-%] | [μm] | [wt.-%] | [wt.-%] |
| 2.9 | — | 0 | 20 | 58.3 | 29.6 |
| | | | 49 | | |
| 2.10 | isophtalaldehyde | 0.25 | 22 | 83.4 | 9.1 |
| | | | 46 | | |
| 2.11 | isophtalaldehyde | 0.50 | 14 | 95.8 | 3.0 |
| | | | 25 | | |
| 2.12 | isophtalaldehyde | 0.75 | 17 | 95.8 | 10.7 |
| | | | 46 | | |
| 2.13 | isophtalaldehyde | 1.0 | 10 | 96.1 | 8.0 |
| | | | 18 | | |
| 2.14 | isophtalaldehyde | 1.25 | 6 | 96.1 | 0.5 |
| | | | 10 | | |

The results of Table 1 and Table 2 show that the microcapsules obtained in the presence of 0.25 wt.-% and more of enabler have a better encapsulation efficiency than if the enabler is not present. At 0.5 wt.-% and more, not only the encapsulation efficiency is improved but also the size of the microcapsules is in the preferred range.

The results also show that the optimal conditions for cross-linking the microcapsules with both glutaraldehyde and transglutaminase are met at an interfacial enabler concentration of 1.25 wt.-%, based on the total weight of the core composition. Under these particular conditions, there is no significant difference between the action of both cross-linking agents, as shown by the similar amount of measured extractable oil in heptane in both cases.

The low level of heptane extractable oil in sample 2.8 compared to the low encapsulation efficiency suggests that the microcapsules obtained with pimelic acid as enabler are less thermally stable that the microcapsules obtained with isophtalaldehyde as enabler.

### EXAMPLE 3: COMPARATIVE EXAMPLE

In Example 4.1, comparative microcapsules were prepared by performing the steps of:
a) Providing 165 g of a core composition consisting of medium chain triglycerides Miglyol 812, ex Oleo;
b) Providing a first aqueous phase by dissolving 16.5 g Type B fish gelatin in 148.5 g of deionized water at a temperature of 40° C.;
c) Providing a second aqueous phase by dissolving 1.6 g of carboxymethylcellulose in 77.9 g of deionized water at a temperature of 40° C.;
d) Adding the core composition to the first aqueous phase under stirring at 300 rpm, by using a stir paddle, and maintaining the stirring for 30 minutes in order to obtain an emulsion;
e) Adding the second aqueous phase to the emulsion obtained in step d) under stirring;
f) Adding 475 g of deionized water, pre-heated to 40° C., while maintaining stirring;
g) Adjusting the pH of the emulsion to the coacervation pH of 5.2=0.5 by using a 50 wt.-% citric acid solution and cooling the emulsion to 28±1° C. at a rate of 1° C. per 5 min in order to form a slurry of core-shell microcapsules;
h) Cooling the slurry formed in g) to a temperature of 20±5° C.;
i) Adding 0.26 g of glutaraldehyde and letting the slurry under stirring overnight (about 15 hours) in order to obtain a slurry of cross-linked core-shell microcapsules;
j) Separating the microcapsules obtained in step j) from the aqueous phase by flotation in order to obtain a cake of microcapsules;
k) Drying the cake obtained in step j) in a vacuum oven dryer at about 80° C. or in a fluid bed dryer at about 70° C., in order to obtain free-flowing, so-called “blank” microcapsules containing medium chain triglycerides as core composition with no fragrance;
l) Transferring 56.8 g of blank microcapsules into a low shear mixer, such as a ribbon blender;
m) Adding 5 g of deionized water over the whole surface of the blank microcapsules;
n) Switching on the blender at 60±5 rpm and leaving to mix for 10-15 minutes until the mix is homogeneous, in order to obtain hydrated blank microcapsules;
o) Switching off the blender and adding 38.2 g of fragrance composition over the whole surface of the hydrated blank microcapsules obtained in step n);
p) Switching on the blender at 60±5 rpm and leaving to mix for 10-15 minutes;
q) Switching off the blender, closing the lit and leaving the microcapsules to absorb the fragrance oil until a dry powder of fragranced microcapsules is obtained, in order to form a dry powder of fragranced microcapsules;

The size of the microcapsules was limited by sieving the powder obtained in step q) through a 150 micrometer screen. The actual fragrance content of the microcapsules was 50 wt.-%, based on the total weight of the microcapsule.

### EXAMPLE 4: OLFACTIVE EVALUATION UNDER AXILLA

The slurries of microcapsules obtained in Example 1.1 and in Example 3.1 were incorporated in a standard, unperfumed deodorant roll-on formulation, so that the level of fragrance in the formulation was 0.1 wt.-%, based on the total weight of the formulation. The formulation was let to macerate for two days.

The olfactive evaluation was performed by a panel of female, non-trained assessors, directly from the axilla. The assessment was performed when the deodorant had been freshly applied, and then after 2 hours, 6 hours and 10 hours through one layer of cloth. After 10 hours the participants were asked to move both arms forwards and backwards in one motion and then rate the fragrance intensity, firstly through one layer of cloths and secondly directly from the skin.

Allocation of which sample was applied to which arm (left or right) was carried out according to a predetermined randomization and the assessors were always asked to assess their left underarm first.

The perceived intensity of the fragrance was assessed by the non-trained assessors using a 0-10 scale. The assessors are told that 0=very weak and 10=very strong.

Each sample was assessed by 56 female, non-trained assessors.

The data were analyzed using a paired sample t-test with the confidence level set at 95%. An individual paired t-test test was performed for each time point. The results are reported in Table 3. Where the same letter is shown in the “significance of differences” column there are no statistically significant differences between the relevant figures at a 95% confidence level.

TABLE 3
Fragrance intensity scores under axilla

| | | Perceived Fragrance | Significance of |
|---|---|---|---|
| Example | Time [hours] | Intensity | Differences |
| 1.1 | 0 | 4.8 | A |
| 2.1 | 0 | 4.5 | B |
| 1.1 | 2 | 3.5 | A |
| 2.1 | 2 | 3.3 | A |
| 1.1 | 6 | 2.5 | A |
| 2.1 | 6 | 2.2 | B |
| 1.1 | 10 | 1.6 | A |
| 2.1 | 10 | 1.5 | A |
| 1.1 | 10 (after rubbing) | 1.8 | A |
| 2.1 | 10 (after rubbing) | 1.6 | B |
| 1.1 | 10 (on skin) | 1.7 | A |
| 2.1 | 10 (on skin) | 1.6 | A |

As apparent from Table 3, the microcapsules according to the present invention perform tendentially better than or at least as well as the comparative, non-vegan microcapsules at all stage of the evaluation process.

### EXAMPLE 5: OLFACTIVE EVALUATION ON PAD

The slurries of microcapsules obtained in Example 1.1 and Example 1.2 were incorporated in a standard, unperfumed deodorant roll-on formulation, so that the level of fragrance in the formulation was 0.1 wt.-%, based on the total weight of the formulation. The formulation was let to macerate for two days.

0.6 g of each formulation was applied on a cotton pad with a pipette. The olfactive intensity was assessed by 10 expert panelists on fresh samples and on samples that was applied 6 hours previously.

Each of the 6 hours samples was assessed before and after rubbing one part of the pad against another part of the pad, wherein the first assessment was performed to score the pre-rub intensity and the second assessment was performed to score the post-rub intensity. The scores were given on a scale of 5 units (0=no intensity, 1=very low, 2=low, 3=average, 4=strong and 5=very strong intensity). The results are reported on Table 4.

TABLE 4
Fragrance intensity scores on pad

| Example | Intensity at t = 0 | Pre-rub intensity | Post-rub intensity |
|---|---|---|---|
| 1.1 | 2 | 0 | 3 |
| 1.2 | 2 | 0 | 3 |

The results show that microcapsules cross-linked with transglutaminase perform as well as microcapsules cross-linked with glutaraldehyde.

1. An encapsulated composition comprising at least one core-shell microcapsule, wherein the at least one core-shell microcapsule comprises a core comprising at least one benefit agent and a shell surrounding the core, wherein the shell comprises a first and a second polyelectrolyte which form a complex coacervate, and wherein the microcapsule comprises at least one interfacial enabler.

2. The encapsulated composition according to claim 1, wherein the core consists of a liquid core composition and the interfacial enabler is soluble in the core composition or is derived from a material that is soluble in the core composition.

3. The encapsulated composition according to claim 1, wherein the interfacial enabler is or is derived from a polyfunctional molecule.

4. The encapsulated composition according to claim 3, wherein the interfacial enabler is a cross-linker, cross-linking the first and/or second polyelectrolyte.

5. The encapsulated composition according to claim 1, wherein the first polyelectrolyte is a polyampholyte.

6. The encapsulated composition according to claim 5, wherein the isoelectric point of the polyampholyte is below pH 7.

7. The encapsulated composition according to claim 1, wherein the solubility of the first and second polyelectrolytes is higher than 5 wt.-% water at pH 7±0.5 and at room temperature.

8. The encapsulated composition according to claim 5, wherein the polyampholyte is a protein.

9. The encapsulated composition according to claim 32, wherein the protein originating from a vegan source is selected from soy proteins, pea proteins, rice proteins and hemp proteins, preferably soy proteins.

10. The encapsulated composition according to claim 8, wherein an aqueous solution comprising a nominal percentage of the protein of 5 wt.-% comprises less than 0.1 wt.-% of insoluble material, based on the total weight of the solution.

11. The encapsulated composition according to claim 1, wherein the second polyelectrolyte is a polysaccharide.

12. The encapsulated composition according to claim 33, wherein the polysaccharide comprising carboxylic acid groups is selected from the group consisting of carboxymethylcellulose, gum acacia, alginate, pectin, hyaluronic acid, xanthan gum, gellan gum, and their salts with monovalent alkaline metals, preferably carboxymethylcellulose and sodium carboxymethylcellulose.

13. The encapsulated composition according to claim 34, wherein the carboxymethylcellulose and/or the sodium carboxymethylcellulose have a molecular weight of from 50,000 to 250,000 g/mol, preferably from 75 and a degree of substitution of from 0.5 to 1.5.

14. The encapsulated composition according to claim 1, wherein the weight ratio of the carboxymethylcellulose and/or the sodium carboxymethylcellulose, to the protein is from 0.05 to 0.2.

15. The encapsulated composition according to claim 3, wherein the interfacial enabler is or is derived from a diacid or a dialdehyde.

16. The encapsulated composition according to claim 15, wherein the interfacial enabler is or is derived from a dialdehyde selected from the group consisting of 1,3-benzenedicarbaldehyde, 1,3-cycloxanedicarbaldehyde, cyclopentane-1,3-dicarbaldehyde, furan-2,5-dicarbaldehyde and 1,4-diformylpyperazin.

17. The encapsulated composition according to claim 1, wherein the interfacial enabler is present at a level of from 0.1 to 5 wt.-% based on the weight of the core composition.

18. The encapsulated composition according to claim 1, wherein the weight ratio of the interfacial enabler to the protein is from 0.01 to 0.1.

19. The encapsulated composition according to claim 1, wherein the benefit agent comprised in the core or the core composition is selected from the group consisting of fragrance ingredients, cosmetic ingredients and biologically active ingredients.

20. The encapsulated composition according to claim 1, comprising a plurality of core-shell microcapsules, wherein the volume median diameter of the microcapsules (Dv 50)) is from 1 to 150 μm.

21. A process for preparing an encapsulated composition according to claim 1, the process comprising the steps of:
a) Providing a core composition comprising an interfacial enabler or a material from which an interfacial enabler may be derived;
b) Providing an aqueous phase comprising a first and a second polyelectrolyte;
c) Emulsifying the core composition provided in step a) into the aqueous phase provided in step b) in order to obtain core composition droplets having a volume median diameter of 1 to 100 μm, dispersed in the aqueous phase;
d) Decreasing the pH until the coacervation pH has been reached, forming thereby a slurry of core-shell microcapsules;
e) Optionally: Adding a cross-linking agent, a further cross-linking agent if applicable, and keeping the slurry under stirring, in order to obtain a slurry of cross-linked microcapsules.

22. The process according to claim 21, wherein in step b) the aqueous phase is provided at a pH of from 5 to 13.

23. The process according to claim 21, wherein in step b) the protein is dissolved in the aqueous phase at a temperature of 45±5° C.

24. The process according to claim 21, wherein in step b) the polysaccharide is dissolved in the aqueous phase at a temperature of 45±5° C.

25. The process according to claim 21, wherein in step c) the emulsion is obtained at a temperature of from 30 to 60° C.

26. The process according to claim 21, wherein in step e) the cross-linking agent, the further cross-linking agent if applicable, is added at a temperature of from 30 to 60° C. and the reaction conducted over 1 to 20 hours.

27. The process according to claim 21, wherein in step e) the cross-linking agent, the further cross-linking agent if applicable, is selected from the group consisting of glutaraldehyde and transglutaminase.

28. (canceled)

29. (canceled)

30. A consumer product comprising an encapsulated composition according to claim 1, preferably a fabric care product, a home care product or a personal care product.

31. (canceled)

32. The encapsulated composition according to claim 8, wherein the protein is a protein originating from a vegan source.

33. The encapsulated composition according to claim 11, wherein the polysaccharide is a polysaccharide comprising carboxylic acid groups.

34. The encapsulated composition according to claim 12, wherein the polysaccharide comprising carboxylic acid groups is carboxymethylcellulose and/or sodium carboxymethylcellulose.
